(19) United States
(12) Patent Application Publication
(10) Pub. No.: US 2013/0316298 A1
(43) Pub. Date: Nov. 28, 2013

(54) METHOD AND APPARATUS FOR SUPPORTING DENTAL IMPLANTATION SURGERY

(71) Applicants: KYUSHU UNIVERSITY, NATIONAL UNIVERSITY CORPORATION, Fukuoka-shi (JP); DENSO CORPORATION, Kariya-city (JP)

(72) Inventors: Tatsuya IKEGAMI, Nisshin-shi (JP); Toshihiko KOYAMA, Anjo-shi (JP); Hideki OKUDA, Nagoya (JP); Tadao OHOKA, Nagoya (JP); Makoto HASHIZUME, Kasuga-shi (JP); Riichi OUCHIDA, Fukuoka-shi (JP); Kazutaka TOYODA, Kariya-shi (JP); Byunghyun CHO, Fukuoka-shi (JP)

(73) Assignees: KYUSHU UNIVERSITY, NATIONAL UNIVERSITY CORPORATION, Fukuoka-shi (JP); DENSO CORPORATION, Kariya-city (JP)

(21) Appl. No.: 13/893,437

(22) Filed: May 14, 2013

(30) Foreign Application Priority Data
May 15, 2012 (JP) 2012-111647

(51) Int. Cl. A61B 6/03; A61B 1/00; A61C 8/00; A61B 6/00; A61B 6/14; A61B 1/24

(52) U.S. Cl. CPC A61B 6/032; A61B 6/14; A61B 1/24; A61C 8/0089; A61B 6/52; A61B 1/00009
USPC 433/29; 433/173

(57) ABSTRACT

In a method and apparatus for supporting dental implantation surgery, a three-dimensional CT image of jaws of an object is acquired. A reference site of the jaws and an implantation position of a gum in the jaws are set in the three-dimensional CT image. The implant is implanted at the implantation position. A three-dimensional optical image of an inside of an oral cavity of the object is then produced. The reference site is positionally set in the three-dimensional optical image through recognition of a shape of the reference site in the three-dimensional optical image. A surgical tool is positionally controlled to the implantation position in the oral cavity, based on i) a relationship between the position of the reference site and the implantation position of the gum in the three-dimensional CT image and ii) the position of the reference site in the three-dimensional optical image.

### CROSS-REFERENCE TO RELATED APPLICATION

This application is based on and claims the benefit of priority from earlier Japanese Patent Application No. 2012-111647 filed May 15, 2012, the description of which is incorporated herein by reference.

### BACKGROUND OF THE INVENTION

1. Technical Field of the Invention

The present invention relates to a method and apparatus for supporting dental implantation surgery.

2. Related Art

A dental implant is an artificial dental root implanted In a jaw to retain a crown or support a prosthetic appliance. Parameters, such as size, direction, shape, and the like, of a dental implant are required to be determined on a patient-to-patient basis and according to the conditions of the site where the dental implant is implanted. For example, a patent document JP-A-2009-501036 suggests a method of determining these parameters on a computer-based simulator.

In implanting a dental implant, accurate positioning is required to be performed in the oral cavity of a patient. To this end, a patent document US2009/0253095 A1 suggests a method in which the position of a patient's head is fixed using a guide member to implant a dental implant at a predetermined position using a surgical device that interlocks with the guide member.

However, it has been difficult for an apparatus of the conventional art to appropriately set an implantation position, while simultaneously controlling position of the surgical tool.

### SUMMARY

Hence, it is desired to provide an apparatus for supporting dental implantation surgery, which is able to solve the problem.

As a one aspect of the present disclosure, there is provided an apparatus for supporting dental implantation surgery. The apparatus includes CT (computed tomography) image acquiring means (3) for acquiring a three-dimensional CT image of jaws of an object; a first setting section (7) for setting a reference site of the jaws and an implantation position of a gum in the jaws in the three-dimensional CT image, an implant being implanted at the implantation position of the gum; a three-dimensional optical image acquiring section (11, 21) for acquiring a three-dimensional optical image of an inside of an oral cavity of the object; a second setting section (21) for setting a position of the reference site in the three-dimensional optical image by recognizing a shape of the reference site in the three-dimensional optical image; and a control section (15, 21) for controlling a position of a surgical tool (13) to the implantation position in the oral cavity, based on i) a relationship between the position of the reference site and the implantation position of the gum in the three-dimensional CT image and ii) the position of the reference site in the three-dimensional optical image.

As another aspect of the disclosure, there is provided a method of supporting dental implantation surgery. The method includes steps of: acquiring a three-dimensional CT image of jaws of an object; first setting a reference site of the jaws and an implantation position of a gum in the jaws in the three-dimensional CT image, an implant being implanted at the implantation position of the gum; acquiring a three-dimensional optical image of an inside of an oral cavity of the object; second setting a position of the reference site in the three-dimensional optical image by recognizing a shape of the reference site in the three-dimensional optical image; and controlling a position of a surgical tool (13) to the implantation position in the oral cavity, based on i) a relationship between the position of the reference site and the implantation position of the gum in the three-dimensional CT image and ii) the position of the reference site in the three-dimensional optical image.

According to the apparatus and method, an appropriate implantation position can be set based on the three-dimensional CT image, and the surgical tool can be controlled so as to be brought to the implantation position.

### BRIEF DESCRIPTION OF THE DRAWINGS

In the accompanying drawings:

FIG. 1 is a block diagram illustrating a configuration of an apparatus for supporting dental implantation surgery, according to an embodiment of the present invention;

FIG. 2 is a flow diagram illustrating a series of processing steps performed by the apparatus;

FIG. 3 is a flow diagram illustrating the series of processing steps continuing from the flow diagram illustrated in FIG. 2;

FIG. 4 is a flow diagram illustrating the series of processing steps continuing from the flow diagram illustrated in FIG. 3;

FIG. 5 is an explanatory diagram illustrating a three-dimensional CT (computed tomographic) image;

FIG. 6 is an explanatory diagram illustrating a three-dimensional CT image superposed with an implant, an operation prohibited area and reference sites;

FIG. 7 is an explanatory diagram illustrating a three-dimensional optical image;

FIG. 8 is an explanatory diagram illustrating a three-dimensional optical image superposed with the implant, the operation prohibited area and the reference sites;

FIG. 9 is a perspective diagram illustrating a configuration including a three-dimensional measuring device, a robot and a surgical tool;

FIG. 10A is an explanatory diagram illustrating a reference site and the position of the surgical tool before movement of the lower jaw; and

FIG. 10B is an explanatory diagram illustrating the reference site and the position of the surgical tool after movement of the lower jaw.

### DETAILED DESCRIPTION OF THE PREFERRED EMBODIMENTS

With reference to the accompanying drawings hereinafter is described an embodiment of the present invention.

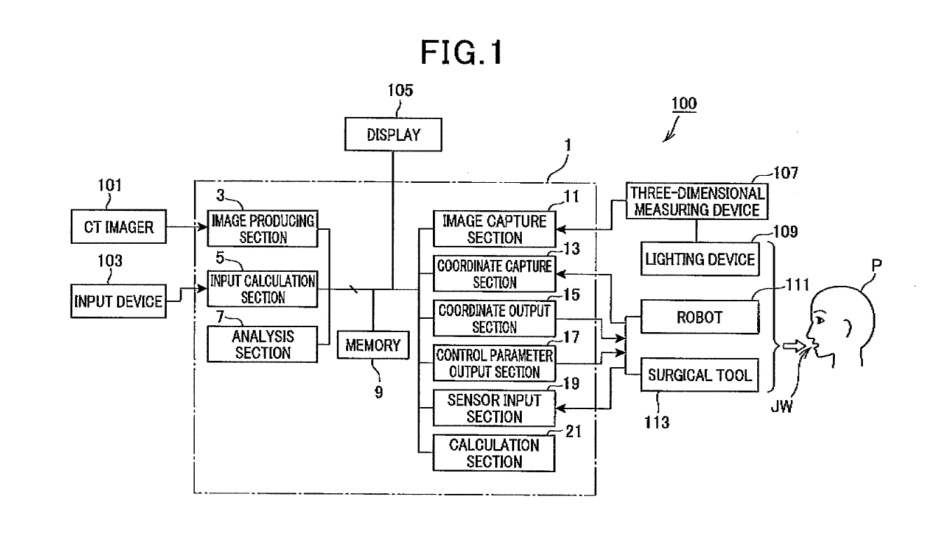

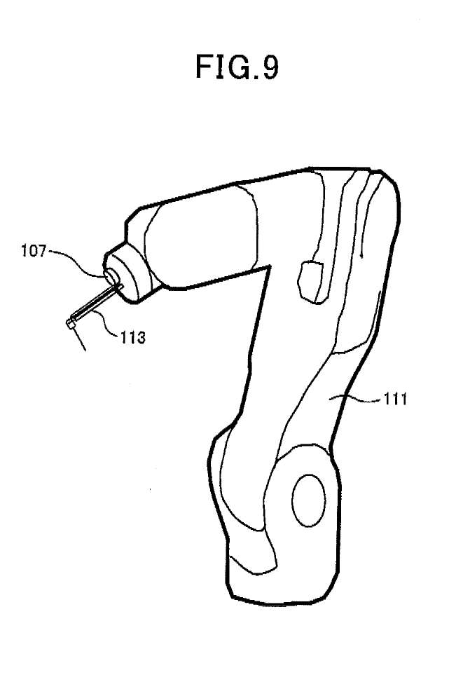

Referring to FIGS. 1 and 9, hereinafter is described a configuration of an apparatus 1 for supporting dental implantation surgery (hereinafter also just referred to as “apparatus 1”).

FIG. 1 is a block diagram pictorially outlining a configuration of the apparatus 1. The apparatus 1 includes an image producing section 3, input calculation section 5, analysis section 7, memory 9, image capture section 11, coordinate capture section 13, coordinate output section 15, control parameter output section 17, sensor input section 19 and calculation section 21. These components of the apparatus 1 perform a series of processing steps that will be described later. These components are realized by installing a program in a well-known computer. The program is stored in the memory 9. Alternatively, the program may be stored in other various well-known storage media.

The apparatus 1 configures a system 100 for supporting dental implantation surgery, together with a CT (computed tomography) imager (or scanner) 101, input device 103, display 105, so three-dimensional measuring device 107, lighting device 109, robot 111 and surgical tool 113.

The CT imager 101 is a well-known device that can pick up a CT image (e.g., CT image in a horizontal cross-sectional plane; more practically, a CT image of each of a plurality of slices) of the jaws JW with gums of a patient P (i.e., an object being subjected to dental implantation surgery). The image producing section 3 (CT image acquiring means) of the apparatus 1 acquires a CT image picked up by the CT imager 101.

Alternatively, the CT imager 101 may be a three-dimensional CT scanner which uses a multiple-row X-ray detector whose multiple-row X-ray elements output a plurality of sets of X-ray projection data at the same time for each of projection angles. The plurality of sets of X-ray projection data are thus subjected to a three-dimensional reconstruction to provide three-dimensional CT image data.

The input device 103 is a well-known inputting means, such as a keyboard, a computer mouse, a touch panel, or other various switches, through which a user can input data. The input calculation section 5 of the apparatus 1 acquires input of the input device 103.

The display 105 is a well-known image display device, such as a liquid crystal display, an organic EL (electroluminescence) display or a cathode-ray tube display. The display 105 displays an image, on the basis of an image signal outputted from the apparatus 1.

The three-dimensional measuring device 107 is a camera that can pick up an optical image (i.e., a visible image) of the inside of the oral cavity of a patient. The lighting device 109 is a known light that can illuminate the inside of the oral cavity of a patient. As shown in FIG. 9, the three-dimensional measuring device 107 is mounted to an extreme end of the robot 111, together with the surgical tool 113. The measuring device 107 and the surgical tool 113 have a constantly fixed relative positional relationship.

FIG. 9 is a perspective diagram illustrating a configuration including the three-dimensional measuring device 107, the robot 111 and the surgical tool 112. As shown in FIG. 9, the robot 111 is a well-known robot having a multijoint arm. The multijoint arm has an extreme end to which the measuring device 107 and the surgical tool 113 are mounted. The robot 111 is able to freely move the measuring device 107 and the surgical tool 113, which are mounted to the extreme end, in a three-dimensional space. The movement of the robot 111 is controlled based on a three-dimensional coordinate system. When a specific coordinate is inputted from the coordinate output section 15 of the apparatus 1, the robot 111 moves the measuring device 107 and the surgical tool 113 to a position corresponding to the specific coordinate. Further, the robot 111 outputs the coordinate of the surgical tool 113 at the time to the coordinate capture section 13 of the apparatus 1.

The surgical tool 113 is a drill. The control parameter output section 17 of the apparatus 1 transmits a signal for instructing the number of revolutions to the surgical tool 113. In response, the surgical tool 113 rotates the drill with the number of revolutions corresponding to the signal. The surgical tool 113 outputs the number of revolutions of the drill and the torque applied to the drill at the time to the sensor input section 19 of the apparatus 1.

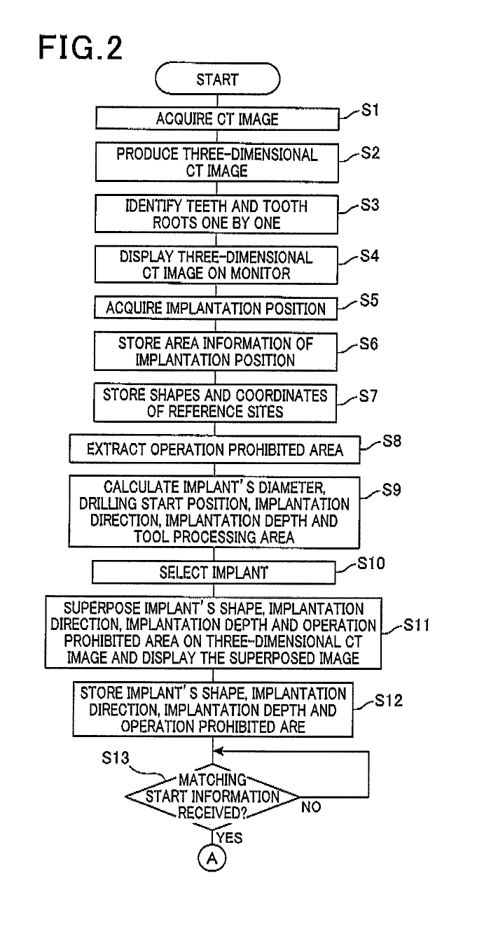

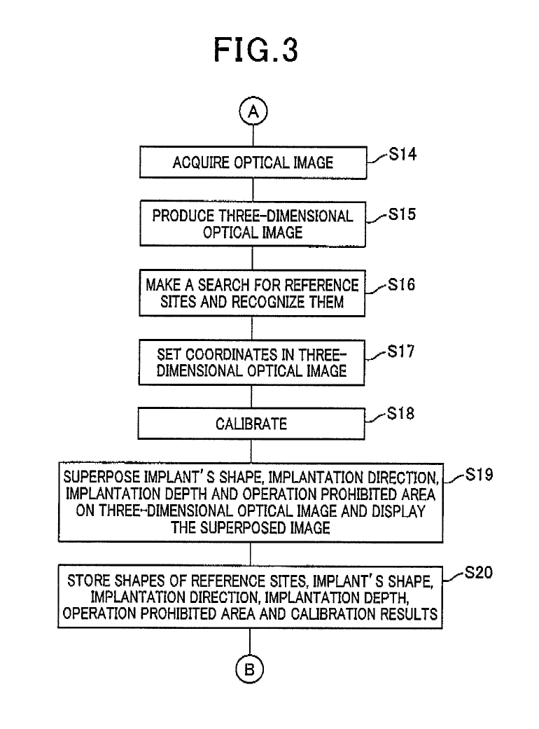

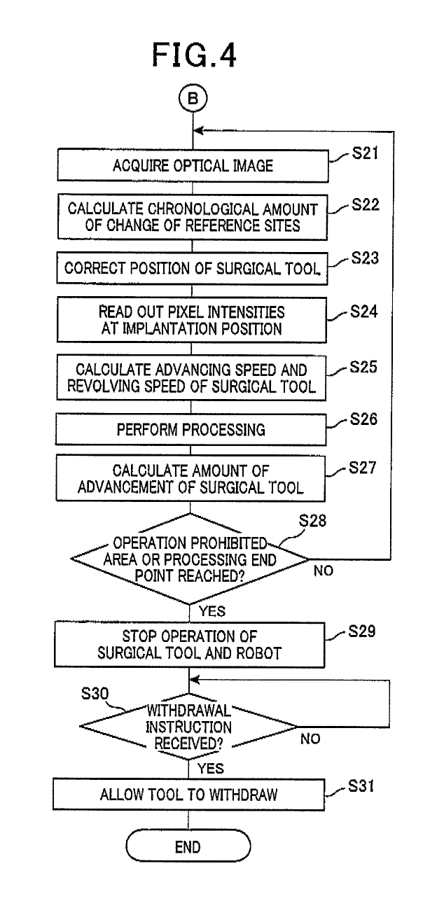

Referring to FIGS. 2 to 4, hereinafter are described a series of processing steps performed by the apparatus 1.

FIGS. 2 to 4 show a flow diagram of the series of processing steps performed by the apparatus 1. As shown in FIG. 2, at step Si, the image producing section 3 acquires a CT image of the jaws JW of a patient P, which is picked up by the CT imager 101. The CT image is picked up in a horizontal cross-sectional plane. At step Si, the image producing section 3 acquires two or more CT images of slices of the jaws, each slice having an imaging plane at a slightly different position (level). The CT imager 101 can be controlled by the apparatus 1.

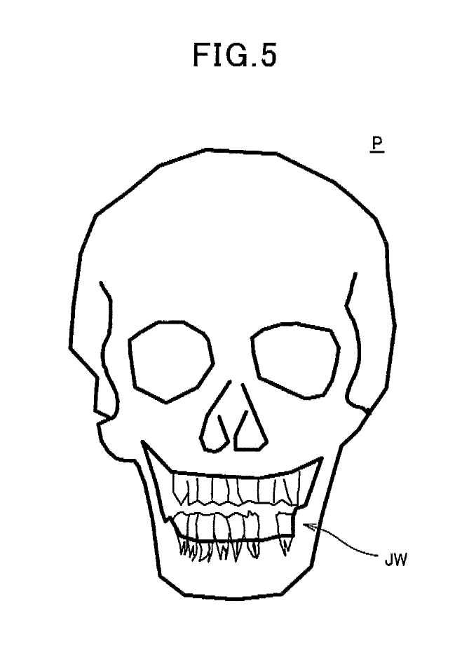

At step S2, the image producing section (three-dimensional CT image producing means) 3 produces a three-dimensional CT image using a well-known image processing technique, on the basis of the two or more CT images acquired at step S1. The three-dimensional CT image expresses the jaws JW of a patient P in a three-dimensional manner. An example of the three-dimensional CT image is shown in FIG. 5.

In cases where the CT imager 101 provides three-dimensional CT image data, the step S2 can be omitted.

At step S3, the image producing section 3 identifies teeth and tooth roots one by one in the three-dimensional CT image produced at step S2, using an image recognition technique.

At step S4, the image producing section 3 displays the three-dimensional CT image on the display 105.

At step S5, the input calculation section 5 acquires an implantation position at which an implant is implanted. The implantation position is inputted by a user via the input device 103. Alternatively, the implantation position may be automatically determined by the apparatus 1 based on the three-dimensional CT image.

At step S6, in the three-dimensional CT image, the analysis section 7 calculates information in the vicinity of the implantation position (area information) and stores the calculated information in the memory 9. The area information includes the size of the gaps between the teeth, the shape of the bone, and the pixel intensities of the portion corresponding to the bone, and the like, near the implantation position.

At step S7, the analysis section (first setting means) 7 sets an implantation position and three reference sites in the three-dimensional CT image. The implantation position is the one that has been acquired at step S5. The reference sites may each correspond to a tooth having a characteristic shape. It is preferred that the three reference sites be set as three teeth which are different in height levels in the jaw from each other and which are distant from each other. Then, the analysis section 7 stores, in the memory 9, the implantation position, the shapes of the reference sites and coordinates indicating the positions of the reference sites in the three-dimensional CT image (hereinafter referred to as coordinates in the three-dimensional CT image). The number of the reference sites is not limited to three but may be a different number of more than three (e.g., 4, 5, 6, etc.)

At step S8, the analysis section 7 extracts an operation prohibited area. The operation prohibited area corresponds to an area where nerves or blood vessels are present. The analysis section 7 is able to extract the operation prohibited area based on the shapes and the pixel intensities, which are specific to nerves and blood vessels, using an image recognition technique.

At step S9, the analysis section 7 calculates parameters including the diameter of an implant to be implanted, a drilling start position, an implantation direction, an implantation depth and a tool processing area. These parameters are calculated according to a predetermined program on the basis of the area information that has been stored at step S6 and the operation prohibited area that has been extracted at step S8. In this case, the parameters are calculated such that the implant will not interfere with the adjacent teeth and that an end of the implant will not reach the operation prohibited area.

At step S10, the analysis section 7 selects an implant suitable for the parameters calculated at step S9. The memory 9 is stored, in advance, with a library of implants having various shapes and sizes. Thus, the analysis section 7 is able to select an implant suitable for the parameters calculated at step S9.

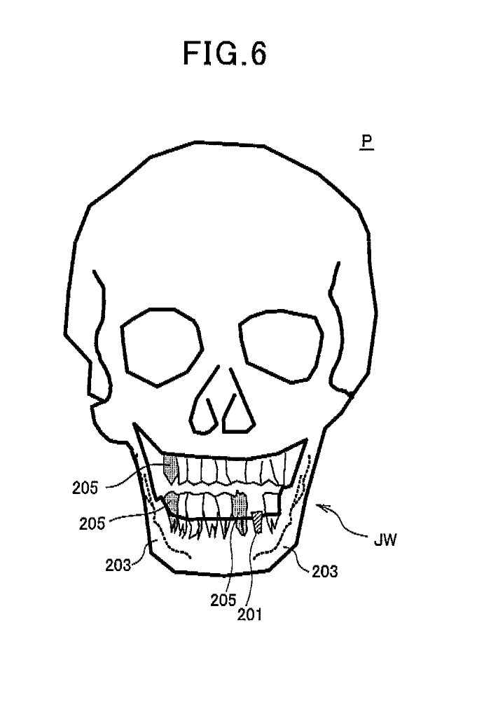

At step S11, the analysis section 7 superposes the shape of the implant selected at step S10, the operation prohibited area extracted at step S8 and the reference sites, into the three-dimensional CT image. Then, the analysis section 7 displays the superposed image on the display 105. The position of the implant displayed here is the implantation position that has been set at step S7. Also, the implantation direction and the implantation depth displayed here are those which have been calculated at step S9. Further, the reference sites displayed here are those which have been set at step S7. An example of the superposed image displayed at step S11 is shown in FIG. 6. The superposed image includes an implant 201 (shape of implant), an operation prohibited area 203 and three reference sites 205.

At step S12, the analysis section 7 stores, in the memory 9, the shape of the implant selected at step S10, the implantation direction and the implantation depth calculated at step S9, and the operation prohibited area calculated at step S8.

At step S13, the input calculation section 5 determines whether or not matching start information has been received. The matching start information corresponds to a predetermined signal inputted by a user via the input device 103. If the matching start information has been received, control proceeds to step S14. If the matching start information has not been received, control returns to step S13.

At step S14, the image capture section (optical image acquiring means) 11 acquires an optical image of the inside of the oral cavity of a patient, which is picked up by the three-dimensional measuring device 107. When this image is picked up, the inside of the oral cavity is illuminated by the lighting device 109. The measuring device 107 and the lighting device 109 can be controlled by the apparatus 1. Two or more such optical images are picked up by changing the imaging position and angle.

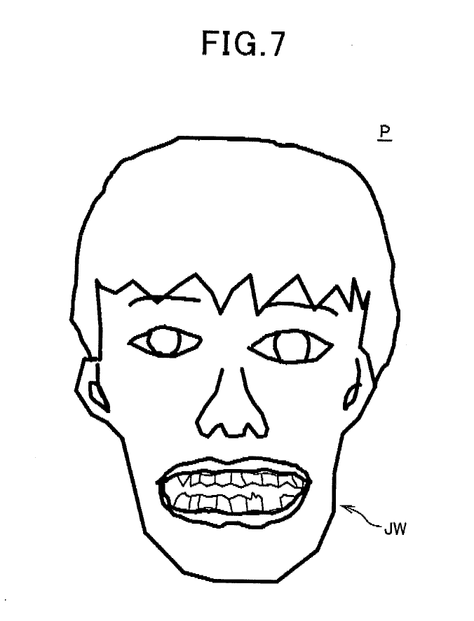

At step S15, the calculation section (three-dimensional optical image producing means) 21 produces a three-dimensional optical image, on the basis of the two or more optical images acquired at step S14, using a well-known image processing technique. The three-dimensional optical image indicates the inside of the oral cavity of a patient in a three-dimensional manner. An example of the three-dimensional optical image is shown in FIG. 7.

At step S16, the calculation section (second setting means) 21 makes a search for the shapes of the three reference sites stored at step S7 and recognizes them, in the three-dimensional optical image produced at step S15, using an image recognition technique. Then, the calculation section 21 sets positions of the three reference sites in the three-dimensional optical image.

At step S17, the calculation section (part of control means) 21 superimposes the three-dimensional CT image over the three-dimensional optical image so that the three reference sites in the former image coincide with the respective three reference sites in the latter image. Then, the calculation section 21 sets a coordinate system in the three-dimensional optical image, using one of the three reference sites as a point of origin. In the coordinate system in the three-dimensional optical image, the implantation position is indicated by a specific coordinate. The specific coordinate allows the positional relationship of the reference sites with respect to the implantation position in the three-dimensional optical image to coincide with that in the three-dimensional CT image.

At step S18, the coordinate system of the robot 111 and the coordinate system that has been set at step S17 are calibrated. Specifically, the following processing is conducted. First, an end of the surgical tool 113 is moved just above one of the three reference sites in the oral cavity of the patient. This movement may be manually conducted by the user or may be automatically conducted by the robot 111. (In the automatic movement, the robot 111 may locate a reference site in the image picked up by the three-dimensional measuring device 107 and move the surgical tool 113 to the located site.) In a state where the end of the surgical tool 113 is brought just above one of the reference sites, the coordinate capture section 13 captures the coordinate in the coordinate system of the robot 111. Then, the calculation section 21 sets the captured coordinate as a point of origin. The coordinate of the point of origin is outputted to the robot 111 by the coordinate output section 15.

After that, the end of the surgical tool 113 is moved just above a second one of the three reference sites. Then, the coordinate capture section 13 captures the coordinate at the time in the coordinate system of the robot 111. Then, the calculation section 21 sets the captured coordinate as a coordinate that corresponds to the second reference site (as a coordinate of the second reference site in the coordinate system set at step S17). Then, the coordinate output section 15 outputs the coordinate to the robot 111.

After that, the end of the surgical tool 113 is moved just above the third one of the three reference sites. Then, the coordinate capture section 13 captures the coordinate at the time in the coordinate system of the robot 111. Then, the calculation section 21 sets the captured coordinate as a coordinate that corresponds to the third reference site (as a coordinate of the third reference site in the coordinate system set at step S17). Then, the coordinate output section 15 outputs the coordinate to the robot 111. Finally, in the coordinate system of the robot 111, the calculation section 21 converts the coordinate system of the robot 111 so that the coordinates of the three reference sites will be in position as described above.

The calibration will be finished through the processing as described above. Thus, the coordinate system of the robot 111 will coincide with the coordinate system in the three-dimensional optical image, which has been set at step S17.

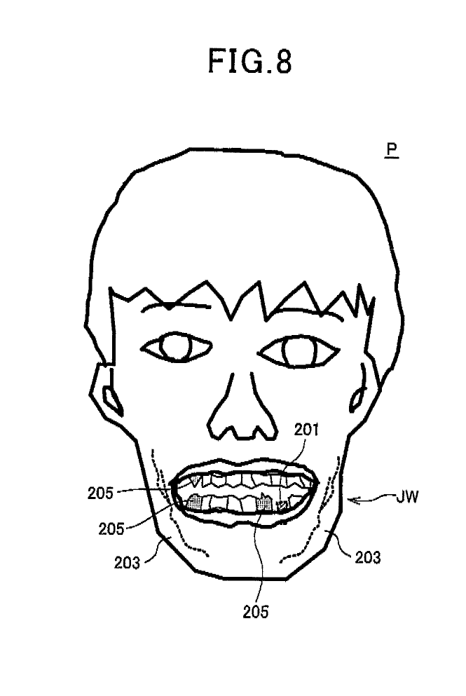

At step S19, the calculation section 21 superposes the shape of the implant selected at step S10, the operation prohibited area extracted at step S8 and the reference sites, into the three-dimensional optical image. Then, the calculation section 21 displays the superposed image on the display 105. The implantation direction and the implantation depth of the implant indicated here are the ones that have been calculated at step S9. An example of the image displayed at step S19 is shown in FIG. 8. The superposed image includes the implant 201 (shape of implant), the operation prohibited area 203 and the three reference sites 205.

At step S20, the calculation section 21 stores, in the memory 9, the shape of the implant selected at step S10, the implantation direction and the implantation depth calculated at step S9, and the operation prohibited area calculated at step S8.

At step S21, a three-dimensional optical image is produced in a manner similar to steps 14 and 15. Specifically, the apparatus 1 updates, as needed, the three-dimensional optical image every time step 21 is performed.

At step S22, the calculation section (chronological change detecting means) 21 recognizes the reference sites in the three-dimensional optical image acquired at the immediately preceding step 21. Then, the calculation section 21 calculates an amount of chronological change in the positions of the reference sites, i.e. from the positions acquired at step S17 to the positions recognized at the present step S22. Specifically, the apparatus 1 calculates a chronological position change of the reference sites in the three-dimensional optical image. For example, the chronological position change of the reference sites is caused by the physical movement or the like of the patient's body.

At step S23, the calculation section (correcting means) 21 corrects the position of the surgical tool 113 (implantation position), based on the amount of change calculated at step S22. For example, when the coordinate of the implantation position acquired at step S17 in the three-dimensional optical image is (x, y, z) and the amount of change acquired at step S22 is (Δx, Δy, Δz), the implantation position is corrected to (x+Δx, y+Δy, z+Δz).

At step S24, the calculation section (operating condition setting means) 21 reads out pixel intensities at the implantation position corrected at step 23, in the three-dimensional optical image that has been produced at step S21. The pixel intensities have a correlation to the hardness of the bone at the implantation position. Specifically, as the pixel intensities have a higher degree, the bone has a higher degree of hardness.

At step S25, the calculation section 21 calculates an advancing speed and a revolving speed of the surgical tool 113 (operating conditions of surgical tool), which are suitable for the pixel intensities read out at step S24. The advancing speed here refers to a speed of sending the drill towards the bone, while the revolving speed here refers to the number of revolutions of the drill. The memory 9 of the apparatus 1 includes a map that outputs an advancing speed and a revolving speed upon input of a degree of pixel intensities. The calculation section 21 calculates an advancing speed and a revolving speed using the map. As the inputted intensities have a higher degree (i.e. as the bone has a higher degree of hardness), the map allows the advancing speed and the revolving speed to become lower. The control parameter output section 17 outputs the calculated advancing speed and revolving speed to the robot 111 and the surgical tool 113. Thus, the robot 111 and the surgical tool 113 are operated according to the advancing speed and the revolving speed calculated as above.

At step 26, the coordinate output section (part of control means) 15 outputs the implantation position corrected at step S23 to the robot 111 and actuates the robot 111 so that the position of the surgical tool 113 coincides with the implantation position. Then, the surgical tool 113 is permitted to perform processing (drill a hole at the implantation position) for a predetermined period. The processing is performed using the advancing speed and the revolving speed calculated at step 25. Further, in the processing, the robot 111 and the surgical tool 113 detect a resistance in the revolution and a resistance in the advancement, and output the detected resistances to the sensor input section 19.

At step S27, the calculation section 21 calculates the depth of the drilling performed at step S26 (product of the advancing speed and the processing period). Then, the calculation section 21 adds the calculated product to a cumulative drilling amount up to then to thereby calculate the latest cumulative drilling amount.

At step S28, the calculation section 21 determines whether or not at least either one of the following conditions has been met.

(Condition 1): The cumulative drilling amount calculated at step S27 has become equivalent to an amount that allows the surgical tool 113 to reach the operation prohibited area that has been stored at step S20.

(Condition 2): The cumulative drilling amount calculated at step S27 has become equivalent to an amount that allows the surgical tool 113 to reach a preset processing end point.

If at least either one of the two conditions is met, control proceeds to step S29. If neither of the conditions is met, control returns to step S21.

At step S29, the calculation section 21 stops the operation of the surgical tool 113 and the robot 111.

Then, at step S30, the calculation section 21 determines whether or not a withdrawal instruction has been inputted via the input device 103. If a withdrawal instruction has been inputted, control proceeds to step S31, but, if not, control returns to step S30.

At step S31, the coordinate output section 15 outputs a coordinate to the robot 111, which will allow the surgical tool 113 to move away from the implantation portion. As a result, the surgical tool 113 withdraws from the implantation portion.

[Effects Exerted by the Apparatus 1 for Supporting Dental Implantation Surgery]

(1) The apparatus 1 is able to set an implantation position on the basis of a three-dimensional CT image and control the surgical tool 113 so as to be positioned at the implantation position.

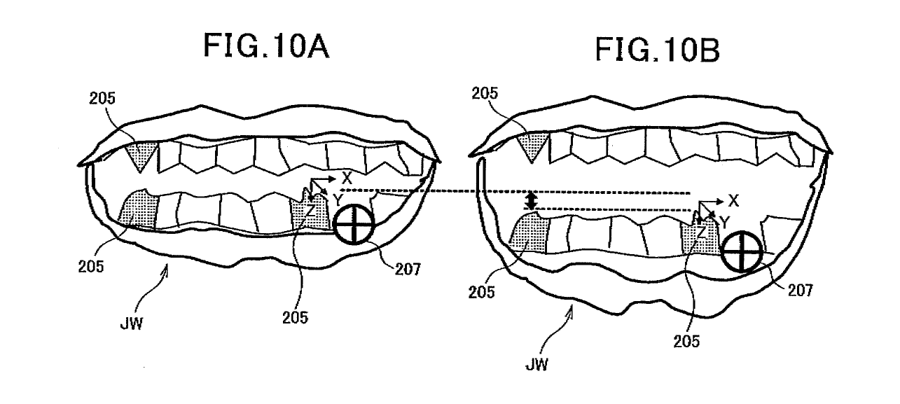

(2) The apparatus 1 updates, as needed, a three-dimensional optical image. If the reference sites in the three-dimensional optical image change their positions with time, the apparatus 1 corrects the position of the surgical tool 113 according to the chronological position change of the reference sites. Accordingly, in the event there is a change in the position or direction of the patient's head during the surgery, the position of the surgical tool 113 can be maintained at an appropriate position. For example, the patient's lower jaw JW at a position shown in FIG. 10A may move to a position shown in FIG. 10B (in which the reference sites have moved downward compared to the position shown in FIG. 10A). In such a case, the position (drilling start position) 207 of the surgical tool 113 relative to the reference sites can be steadily maintained.

(3) The apparatus 1 sets the operating conditions (advancing speed and revolving speed) of the surgical tool 113 based on the pixel intensities at the implantation position in the three-dimensional CT image. Accordingly, appropriate operating conditions can be set so as to be suitable for the hardness of the bone.

(4) The apparatus 1 is able to acquire area information and an operation prohibited area in the three-dimensional CT image. Then, based on the acquired area information and operation prohibited area, the apparatus 1 is able to set a diameter of an implant, a drilling start position, an implantation direction, an implantation depth and a tool processing area.

The present invention may be embodied in several other forms without departing from the spirit thereof. The embodiment described so far are therefore intended to be only illustrative and not restrictive, since the scope of the invention is defined by the appended claims rather than by the description preceding them. All changes that fall within the metes and bounds of the claims, or equivalents of such metes and bounds, are therefore intended to be embraced by the claims.

What is claimed is:

1. An apparatus for supporting dental implantation surgery, comprising:
CT (computed tomography) image acquiring means for acquiring a three-dimensional CT image of jaws of an object;
first setting means for setting a reference site of the jaws and an implantation position of a gum in the jaws in the three-dimensional CT image, an implant being implanted at the implantation position of the gum;
three-dimensional optical image acquiring means for acquiring a three-dimensional optical image of an inside of an oral cavity of the object;
second setting means for setting a position of the reference site in the three-dimensional optical image by recognizing a shape of the reference site in the three-dimensional optical image; and
control means for controlling a position of a surgical tool to the implantation position in the oral cavity, based on i) a relationship between the position of the reference site and the implantation position of the gum in the three-dimensional CT image and ii) the position of the reference site in the three-dimensional optical image.

2. The apparatus of claim 1, wherein the CT image acquiring means comprises
CT image receiving means for receiving a plurality of the CT images of the jaws; and
a three-dimensional CT image producing means for producing the three-dimensional CT image from the plurality of the CT images received, and
the three-dimensional optical image acquiring means comprises
optical image receiving means for receiving a plurality of the optical images; and
three-dimensional optical image producing means for producing the three-dimensional optical image from the plurality of the optical images received.

3. The apparatus of claim 1, wherein the reference site is plural in number.

4. The apparatus of claim 1, wherein the three-dimensional optical image producing means has the capability to update three-dimensional optical image at regular intervals, and
the apparatus comprises
chronological change detecting means for detecting chronological positional changes of the reference site in the three-dimensional optical image; and
correcting means for correcting the position of the surgical tool depending on the temporal positional changes of the reference site.

5. The apparatus of claim 4, comprising
operating condition setting means for setting an operating condition of the surgical tool based on pixel intensities of the implantation position in the three-dimensional CT image.

6. The apparatus of claim 1, comprising
operating condition setting means for setting an operating condition of the surgical tool based on pixel intensities of the implantation position in the three-dimensional CT image.

7. The apparatus of claim 2, comprising
operating condition setting means for setting an operating condition of the surgical tool based on pixel intensities of the implantation position in the three-dimensional CT image.

8. The apparatus of claim 3, comprising
operating condition setting means for setting an operating condition of the surgical tool based on pixel intensities of the implantation position in the three-dimensional CT image.

9. The apparatus of claim 2, wherein the reference site is plural in number.

10. The apparatus of claim 4, wherein the reference site is plural in number.

11. A method of supporting dental implantation surgery, comprising steps of:
acquiring a three-dimensional CT image of jaws of an object;
first setting a reference site of the jaws and an implantation position of a gum in the jaws in the three-dimensional CT image, an implant being implanted at the implantation position of the gum;
acquiring a three-dimensional optical image of an inside of an oral cavity of the object;
second setting a position of the reference site in the three-dimensional optical image by recognizing a shape of the reference site in the three-dimensional optical image; and
controlling a position of a surgical tool to the implantation position in the oral cavity, based on i) a relationship between the position of the reference site and the implantation position of the gum in the three-dimensional CT image and ii) the position of the reference site in the three-dimensional optical image.

12. The method of claim 11, comprising steps of:
detecting chronological positional changes of the reference site in the three-dimensional optical image updated at regular intervals; and
correcting the position of the surgical tool depending on the temporal positional changes of the reference site.

13. A computer-readable program readably stored in a memory by a computer, the program having the capability to enable the computer to function as:
acquiring a three-dimensional CT image of jaws of an object;
first setting a reference site of the jaws and an implantation position of a gum in the jaws in the three-dimensional CT image, an implant being implanted at the implantation position of the gum;
acquiring a three-dimensional optical image of an inside of an oral cavity of the object;
second setting a position of the reference site in the three-dimensional optical image by recognizing a shape of the reference site in the three-dimensional optical image; and
controlling a position of a surgical tool to the implantation position in the oral cavity, based on i) a relationship between the position of the reference site and the implantation position of the gum in the three-dimensional CT image and ii) the position of the reference site in the three-dimensional optical image.
